(19) United States
(12) Plant Patent Application Publication
(10) Pub. No.: US 2015/0271964 P1
(43) Pub. Date: Sep. 24, 2015

(54) Almond tree named 'Kester'

(50) ;

(71) Applicant: The Regents of the University of California, Oakland, CA (US)

(72) Inventors: Thomas GRADZIEL, Davis, CA (US); Mario VIVEROS, Bakersfield, CA (US); Dale KESTER, Davis, CA (US)

(73) Assignee: The Regents of the University of California, Oakland, CA (US)

(21) Appl. No.: 13/999,776

(22) Filed: Mar. 19, 2014

(51) Int. Cl. A01H 5/00

(52) U.S. Cl. PLT/155

(57) ABSTRACT

A new almond variety (Prunus dulcis) designated as ‘Kester’ is provided that is well-suited for serving as a pollenizer of the widely-grown ‘Nonpareil’ variety. ‘Kester’ is cross-compatible with ‘Nonpareil’ and has good bloom overlap with this variety, making ‘Kester’ highly complementary to ‘Nonpareil’ in production systems. ‘Kester’ has demonstrated low potential for Noninfectious Bud Failure and other serious afflictions of almond. Further, ‘Kester’ displays desirable tree and nut qualities, along with exceptional yields.

### LATIN NAME

Botanical/commercial classification: Prunus dulcis new almond cultivar.

### VARIETAL DENOMINATION

The varietal denomination of the claimed almond variety is ‘Kester’.

### BACKGROUND OF THE INVENTION

A major objective of almond breeding programs is the development of new varieties capable of acting as a pollenizer variety. In particular, there is a need for the development of new varieties capable of acting as a pollenizer variety for the California almond variety ‘Nonpareil’ (non-patented in the United States). Because of its high market quality, ‘Nonpareil’ has become the leading California almond variety, presently occupying approximately 40% of total almond acreage.

‘Nonpareil’, like most commercial almond varieties, is self-sterile and requires pollen from cross-compatible varieties for successful seed set. California almond variety ‘Winters’ (U.S. Plant Pat. No. 13,286) was released in 2002 as an early-Nonpareil bloom pollenizer to complement well-established late-bloom pollenizers. The California almond cultivar ‘Carmel’ (U.S. Plant Pat. No. 2,641) is currently the second most widely planted cultivar, currently accounting for approximately 10% of total almond production. However, the increasing incidence of the genetic disorder Noninfectious Bud Failure in the ‘Carmel’ variety has resulted in a dramatic decrease in new plantings of this cultivar and prompted the search for new pollenizer varieties having good kernel quality, productivity, and good overlap with the later ‘Nonpareil’ bloom. The new almond variety ‘Kester’ as described herein is a product of this breeding effort.

### SUMMARY OF THE INVENTION

The present disclosure relates to a new and distinct almond cultivar (Prunus dulcis) that has been denominated as ‘Kester’ and more particularly to such an almond variety that is a cross-compatible pollenizer for the later bloom of the major California almond cv. ‘Nonpareil’. In long-term regional testing, ‘Kester’ has demonstrated low potential for Noninfectious Bud Failure and other serious afflictions of almond, as well as demonstrating high yields of Nonpareil-type kernels, maturing just after ‘Nonpareil’ and so is highly complementary to ‘Nonpareil’ in production systems.

Almond variety ‘Kester’ was originally identified as selection 2-19E and was isolated from a seedling population obtained from a cross between ‘Tardy-Nonpareil’ and ‘Arbuckle’ almond varieties. Based on its promising performance, particularly its consistent bloom overlap with the later bloom of ‘Nonpareil’, its cross compatibility with ‘Nonpareil’, and its desirable tree and nut qualities, and exceptional yields, selection 2-19E was selected as a candidate for release as the variety ‘Kester’.

### BRIEF DESCRIPTION OF THE TABLES

TABLE 1 shows the performance results of a Regional Variety Trial of selection 2-19E (‘Kester’) relative to ‘Carmel’ (a standard ‘Nonpareil’ pollenizer) from Year 18 to Year 27.

TABLE 2 shows the performance results of a Variety Trial of selection 2-19E (‘Kester’) compared with the ‘Nonpareil’ quality/productivity standard and other candidate ‘Nonpareil’ pollenizers from Year 29 to Year 35. In each column, values with the same letter are not significantly different (P<0.05).

### BRIEF DESCRIPTION OF THE DRAWINGS

FIG. 1 illustrates the average bloom data for selection 2-19E and other almond varieties obtained from the McFarland Trial from Year 29 to Year 34. The cross-hatched area indicates the onset of bloom to 100% petal fall. The “F” indicates full bloom. The month and the day are indicated at the top of the figure.

FIG. 2 illustrates the average hullsplit progression data for selection 2-19E and other almond varieties obtained from the McFarland Trial from Year 29 to Year 34. The month and the day are indicated at the top of the figure.

FIG. 3 illustrates various morphological phenotypes of selection 2-19E trees. FIG. 3A illustrates the flowering of selection 2-19E. FIG. 3B illustrates the cropping branch of selection 2-19E.

FIG. 4 illustrates various morphological phenotypes of selection 2-19E kernel and nut. Both FIG. 4A and FIG. 4B illustrate different views of the kernels and the nuts of selection 2-19E.

### DETAILED DESCRIPTION OF THE INVENTION

The original almond seedling population from which almond variety ‘Kester’ was selected was produced by routine crosses made during or prior to Year 1 between ‘Tardy-Nonpareil’, a late blooming mutation of ‘Nonpareil’, as the seed (female) parent and ‘Arbuckle’ as the pollen (male) parent. The crosses were made as part of the Calif. Expt. Stat. Proj. 739-Almond Breeding Project carried out in the Department of Pomology, University of California, Davis.

‘Kester’, which was evaluated under the individual seedling designation number ‘2-19E’, was selected in Year 11 based on its desirable kernel and bloom qualities and was propagated in replicated trials in Field 7 at the Wolfskill Experimental Orchard (WEO) near Winters, Calif. for further evaluation under Project POM-5884-H: Genetic Improvement of Peach and Almond. Based on its promising characteristics, selection 2-19E was submitted to the Foundation Plant Service (FPS) in Year 14 for inclusion into the Foundation Block where it was designated by the Accession Number 03-059-01-91. In Year 15, selection 2-19E was included in the Regional Variety Trials and grown under semi-commercial conditions. These trials were supported by the Almond Board of California (project HORT5-Gradziel), which included plots in Kern County, Butte County (Chico), and San Joaquin County (Delta). Twenty-six trees each of multiple selection and varieties were included in each plot. Data of yield, phenological observations, and tree and nut characteristics have been obtained annually during Year 18 to Year 27 (TABLE 1). A record of the data obtained from these field trials is found in annual reports made to the Almond Board of California for Project HORT5-Gradziel beginning in Year 18.

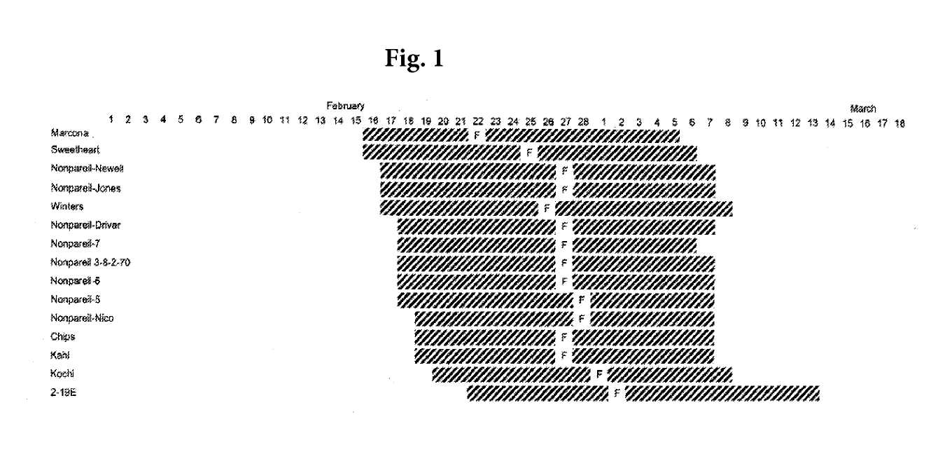

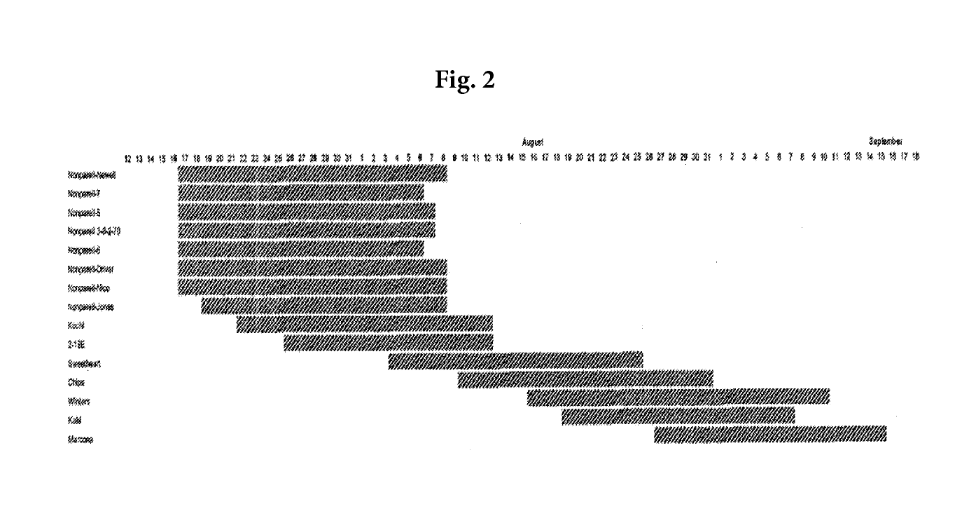

In the trials shown in TABLE 1, although selection 2-19E demonstrated good productivity and desirable tree and kernel characteristics at the Kern County Regional Variety Trial compared to the ‘Cannel’ standard, performance at the Butte and San Joaquin County sites was less promising. In addition, all sites showed evidence of alternate bearing, where a high crop in one year would often be followed by a considerably lower crop the following year. Without wishing to be bound by theory, both the alternate-bearing and the lower productivity in the more northern regional variety trials are thought to be the result of insufficient agronomic inputs for an inherently highly productive selection. Accordingly, a new, replicated, test planting using semi-commercial conditions was established in McFarland, Calif. along with other candidate late-Nonpareil-bloom pollenizers during Years 29-35. The results of these trials are presented in TABLE 2. Average bloom data from these trials for selection 2-19E and other almond varieties is presented in FIG. 1, and average hullsplitting progression data is presented in FIG. 2.

Under these improved orchard management conditions, selection 2-19E continued to demonstrate desirable tree and kernel characteristics and good bloom overlap with the later ‘Nonpareil’ bloom (FIG. 1) while also demonstrating exceptional productivity without evidence for pronounced alternate bearing (TABLE 2). The high similarities between ‘Nonpareil’ and 2-19E kernels may allow their mixing and may thus increase 2-19E crop value, while the harvest time just after ‘Nonpareil’ allows for concurrent and even simultaneous harvest.

### BOTANICAL DESCRIPTION OF THE PLANT

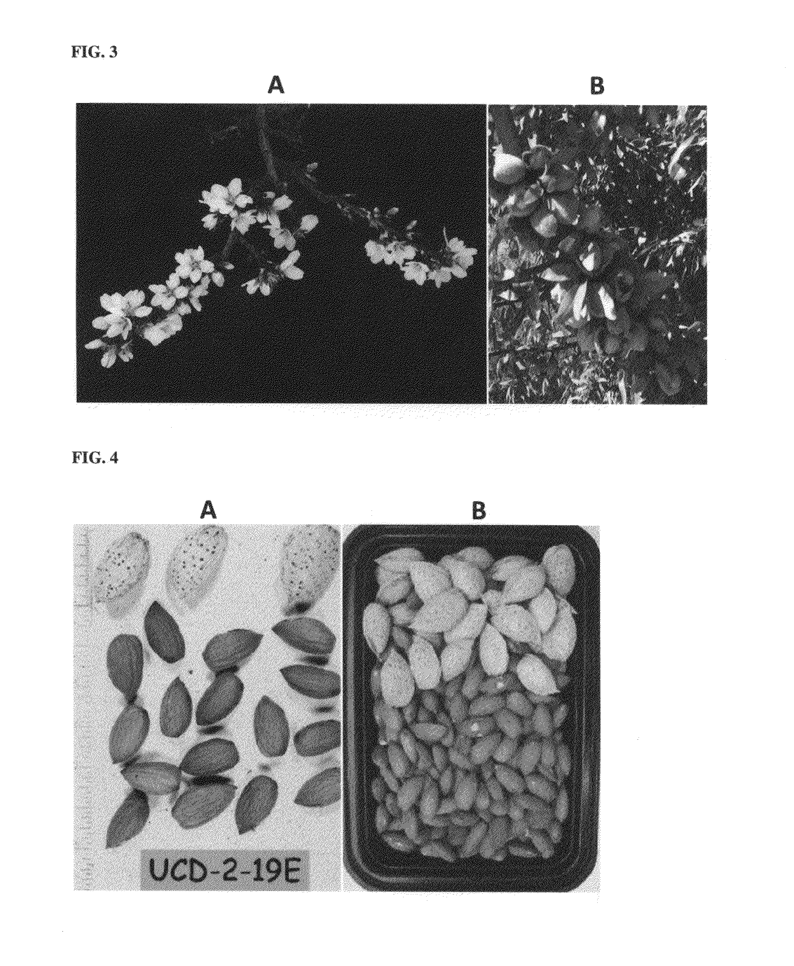

The following is a detailed horticultural description of the new almond variety ‘Kester’ obtained from vegetatively propagated progeny observed during the Year 35 and Year 36 growing seasons. The trees were vegetatively propagated by FPS (Foundation Plant Services) by T-bud propagation onto ‘Lovell’ peach rootstock. The trees were grown at Foundation Plant Service Foundation Stock orchards in Davis, Calif. and at the Plant Pathology Orchards of the University of California located at Davis, Calif. The younger trees for this description were 4^{th} leaf (4 years in the field after nursery propagation), while the older trees were 14^{th} leaf. Color designations are presented with reference to the “Dictionary of Color” by Maerz and Paul, First Edition (1930). All descriptions relate to those observed under early summer growth conditions, with the exception of the bloom and harvested crop descriptions. Bloom and crop performance are summarized from 6 to 12 year regional trials in Butte, Yolo, San Joaquin and Kern counties.
Botanical classification: Prunus dulcis, cv. ‘Kester’. ‘Kester’ is very similar to ‘Nonpareil’ and its seed parent ‘Tardy-Nonpareil’, which is a bud-sport of ‘Nonpareil’, in terms of kernel quality. ‘Kester’ flowers approximately 3 days earlier than ‘Tardy-Nonpareil’ and 3 days later than ‘Nonpareil’, and harvests approximately 10 days after ‘Nonpareil’ and 5 days after ‘Tardy-Nonpareil’. ‘Kester’ has a smaller tree size when compared to ‘Nonpareil’ and ‘Tardy-Nonpareil’, but has comparable production to ‘Nonpareil’ and much higher production than ‘Tardy-Nonpareil’. ‘Kester’ possesses much better kernel quality and much higher yield than its pollen parent ‘Arbuckle’, which is no longer commercially planted.
Tree: The tree size is medium. When compared with the ‘Nonpareil’ variety, the present almond tree is smaller. The shape of the tree is upright to spreading. Tree density may be considered open and the trees exhibit moderate vigor. Generally, the trees exhibit regular bearing and bearing occurs predominantly from spurs which are two years old or older. The trees may tend to alternate-bear after years of very heavy crop.
Trunk: With regard to the tree trunk, the trunk form is generally medium as compared with other almond cultivars. The surface texture of the trunk is average.
Branches: The branches are average as compared with other common almond cultivars. There are relatively few tertiary branches, the present almond tree being similar to ‘Nonpareil’ in this aspect. The shoot surface texture is relatively slender and smooth. Shoot color is medium verdant (20-K-4) with occasional reddish flush (7-J-1). Lenticels are medium in number and small, having an orange-tan color (12-J-10). The shoot form is straight, and have current year laterals typically at the mid to basal-section of longer shoots. Branch length is approximately 10 centimeters to about 30 centimeters (about 4 to about 12 inches), and there are approximately 10 to about 30 nodes present depending upon branch length. The branch internodes are approximately 1 to 2 centimeters in length. Lateral buds are conical in shape, pointed, and develop dark brown scales (8-J-7) by late summer. Double buds may be common at a node. Two to five lateral buds will normally be found, and these will eventually become flower buds. Terminal buds are short, have a pointed shape, and are small and very dark. Lateral buds appear rounded and have a brown color, although this color is not distinctive. Scales are generally dark brown (8-J-7) in late summer, and non-distinctive. Spurs are generally numerous, short, and stubby. The length is approximately 1 cm. On spurs which are one year old, the epidermis takes on a green color, but older spurs are much darker. Two year old wood generally appears somewhat stag brown (14-E-7). Three year old wood generally has many persistent peduncles and from which fruit has been borne on previous years. The color is cinnamon brown (14-H-9). Four year old wood generally shows a dull mustang brown (15-E-8) color which is evident where the epidermis has sloughed off, although this color is not particularly distinctive. Some spurs continue to produce, but many spurs are clusters of persistent peduncles from earlier production.
Leaves: The leaf quantity is abundant. Leaf shape may be considered lanceolate, occasionally elliptical, and tapering to the apex about ⅓ of the way from the apex. The leaf tip has an acuminate shape, and tapering to a more or less acute angle. The leaf base angle is rounded to oblique. The leaves have an average length of approximately 30 to 90 millimeters and a width of approximately 15 to about 25 millimeters. Leaf petioles are medium in both length and thickness. The ratio of petiole length to leaf length is approximately 0.28. The ratio of blade width to blade length is approximately 0.30. Shoot leaves are generally medium in size, flat and substantially uniform. The color on the top surface of shoot leaves is fir green (24-L-5). The color on the bottom surface of shoot leaves is garland green (22-F-5). Leaf margins are crenate with rather shallow crenations. Leaves exhibit venation that is pinnately net veined. With regard to glands, there are usually two glands, alternate on petiole, primarily at the base of the leaf. Glands are small and have a globose form, as well as a russet green color (20-K-1) and tend to slough off with leaf age.
Bloom: The amount of bloom is heavy. The bloom color is white (2-b-1) with light rose (2-J-3) developing at the petal claw. The blooming period typically starts 3-4 days after ‘Nonpareil’, with full bloom typically occurring 3-5 days after ‘Nonpareil’ full bloom. Average bloom data for the present almond variety, along with that of other almond varieties, is illustrated in FIG. 1. The shape of the petal apex is often retuse, occasionally with 1 to 3 shallow clefts on a rounded apex. The petal base forms a broadly cuneate, tapering claw. The configuration of the petal margin is rounded, smooth and slightly crenate at the apex. Petals have a soft velvety texture with cupping at the margins. The majority of flowers contain 5 petals. Typically, less than 3% of the flowers may contain 4 petals or 6 or more petals. The petal size is typically 14 to 20 mm long and 10 to 15 mm wide. The flower size is typically 30 to 40 mm in dimension. FIG. 3A provides an illustration of the flowers of the present variety. The pollen is cross-compatible with ‘Nonpareil’ and so is capable of acting as a pollenizer for this cultivar.
Crop: The present almond tree is a regular bearer, mostly on spurs, and has heavy productivity. Nuts are well-distributed on the tree and on all sizes of wood. The harvest period is early, typically 4 to 7 days after ‘Nonpareil’. The nuts hang well on the tree yet are easy to harvest and easy to hull. FIG. 3B provides an illustration of a cropping branch of the present variety.
Immature fruit: The side view of immature fruit is uniformly elongate, oval. The dorsal edge is slightly curved with more pronounced curving at the apical and basal ends. The ventral edge is uniformly curving along the entire length. The basal end is flat at about 75° to long axis. The apical end has a small, short acute protruding tip. The dorsal view is somewhat rugose, with slight to noticeable asymmetry between sides, particularly for larger fruit. There is a slight ridge, but is barely conspicuous. The ventral view is similar to the dorsal, but slightly less rugose and with a depressed ventral line. The apical end view is rounded with a distinct ventral ridge. The basal view is rounded, with a distinct suture line and often noticeable dorsal and ventral ridges. The base scar is round to oval and has a medium size, and also detaches cleanly. Dehiscence starts at the ventral edge, but eventually extends into the dorsal edge. Pubescence is whitish, very fine, and uniform.
Hull: The outer surface is somewhat rugose and pubescent. The form is uniform and somewhat asymmetrical as with the immature fruit. The longitudinal section form is oval. The thickness is generally 2 to 3mm when dry. The flesh is tough but brittle when dry. The suture is medium and the color is fern green (21-1-7). In terms of dehiscence, the hull opens freely and splitting occurs along the suture. The nut cavity is oval and in terms of adherence, hulls are easily removed from nuts by mechanical hullers. Hullsplit progression in the present almond variety, along with that of other almond varieties, is illustrated in FIG. 2.
Nut: The nut size is medium to large and nuts have a medium light brown color. In terms of form, the length/width is elongated and the width/thickness is medium, similar to ‘Nonpareil’. The shell is a paper-type and is easily cracked. There are few pits, but they are large, deep, and round. The base is ventrally oblique. The stem scar is medium, round to ovate. The apex is blunt, falcate. The wing is broad and thin, and is tapered toward the base. The inner surface is medium brown and the ventral streak is narrow and lighter brown in color. The percentage of kernel to nut is approximately 66%. FIG. 4A and FIG. 4B provides an illustration of the nuts and the kernels of the present variety.
Kernel: The kernel size is similar to ‘Nonpareil’, although slightly smaller. The average kernel length is 21 mm and the average kernel width is 11.5 mm. The average thickness is 7.8 mm and the average weight is 0.94 g. In terms of form, the length/width is medium and the width/thickness is flat, similar to ‘Nonpareil’. The base is ventrally oblique and the stem scar is large, being oval to obtuse. The apex is acuminate, the texture is slightly wrinkled, the pellicle is medium, the pubescence is smooth and veined, the color is a light sayal brown (13-G-9), and the number of doubles is low, being less than 10%. Kernel flavor is sweet, the quality is good, and they are blanchable.
Cultural characteristics: The resistance to insects of the present almond variety is comparable to that of ‘Nonpareil’. The susceptibility to Noninfectious Bud Failure is low. The present variety is moderately susceptible to Hull Rot, Alternaria Leaf Spot, and Scab, being similar to ‘Nonpareil’ in this aspect.

TABLE 1
Regional Variety Trial (Year 18 to Year 27)

| | | | 2-19E | | | | Carmel | | | |
|---|---|---|---|---|---|---|---|---|---|---|
| | | | lbs. | kernel | Crack- | Cumu- | lbs. | kernel | Crack- | Cumu- |
| | | Root- | per | wt. in | out | lative | per | wt. in | out | lative |
| Year | Site | stock | acre | g | (%) | yield | acre | g | (%) | yield |
| 19 | Chico | L | 276 | 1.08 | 46.3 | 276 | 741 | 1.23 | 53.2 | 844 |
| 20 | Chico | L | 1299 | 1.01 | 51.2 | 1576 | 1240 | 1.33 | 56.3 | 2083 |
| 21 | Chico | L | 454 | 1.11 | 43.7 | 2029 | 1260 | 1.36 | 54.5 | 3343 |
| 22 | Chico | L | 1345 | 0.88 | 47.8 | 3374 | 1700 | 1.20 | 56.6 | 5044 |
| 23 | Chico | L | 906 | 1.13 | 46.4 | 4280 | 1934 | 1.37 | ND | 6977 |
| 24 | Chico | L | 1828 | 0.96 | 50.8 | 6108 | 2070 | 1.13 | 52.4 | 9047 |
| 25 | Chico | 1 | 964 | 0.99 | 43.7 | 7072 | 2320 | 1.06 | 50.7 | 11368 |
| 26 | Chico | L | 2023 | 0.99 | 46.2 | 9095 | 2330 | 1.20 | 53.4 | 13697 |
| 27 | Chico | L | 1171 | 1.0 | 43.9 | 10266 | 2830 | 1.1 | 53.0 | 16528 |
| 19 | Delta | N | ND | ND | ND | ND | 114 | 1.41 | 64.9 | 114 |
| 20 | Delta | N | 503 | 0.97 | 55.7 | 503 | 2111 | 1.16 | 60.4 | 2225 |
| 21 | Delta | N | 507 | 1.02 | 50.0 | 1010 | 1893 | 1.24 | 61.2 | 4118 |
| 22 | Delta | N | 1010 | 1.06 | 58.2 | 2020 | 2695 | 1.24 | 58.5 | 6813 |
| 23 | Delta | N | 903 | 1.27 | 63.1 | 2924 | 2538 | 1.27 | ND | 9351 |
| 24 | Delta | N | 1008 | 1.12 | 58.1 | 3932 | 2206 | 1.19 | 58.2 | 11556 |
| 25 | Delta | N | 1245 | 1.16 | 54.8 | 5176 | 2697 | 1.19 | 62.3 | 14253 |
| 26 | Delta | N | 1405 | 1.11 | 54.3 | 6581 | 1861 | 1.25 | 56.2 | 16113 |
| 18 | Kern | N | 341 | 1.18 | 56 | 341 | 634 | 1.496 | 66 | 634 |
| 19 | Kern | N | 963 | 1.17 | 47.5 | 1304 | 1260 | 1.32 | 55.0 | 1893 |
| 20 | Kern | N | 2347 | 0.97 | 52.7 | 3651 | 1944 | 1.25 | 60.8 | 3837 |
| 21 | Kern | N | 1944 | 1.07 | 52.6 | 5595 | 1427 | 1.21 | 67.3 | 5264 |
| 22 | Kern | N | 2496 | 0.88 | 51.2 | 8091 | 1359 | 1.22 | 54.5 | 6623 |
| 23 | Kern | N | 2646 | 1.17 | 60.0 | 10737 | 2534 | 1.43 | ND | 9157 |
| 24 | Kern | N | 3479 | 1.15 | 62.1 | 14216 | 3819 | 1.24 | 62.6 | 12976 |
| 25 | Kern | N | 2434 | 1.01 | 56.6 | 16650 | 3398 | 1.03 | 59.8 | 16375 |
| 26 | Kern | N | 4890 | 1.03 | 66.5 | 21540 | 2651 | 1.24 | 63.2 | 19026 |
| 27 | Kern | N | 1490 | 1.1 | 48.4 | 23030 | 2778 | 1.1 | 58.9 | 21804 |

L—Lovell; N—Nemaguard; ND—No data

TABLE 2
McFarland Variety Trial (Year 29 to Year 35)

| | | | Average | | | | | | | | Cumulative | |
|---|---|---|---|---|---|---|---|---|---|---|---|---|
| Year 29 | No. of | | kernel wt | | Shelling | | Kernel pounds per | | | | kernel yield | |
| Variety | nuts/tree | | (g) | | percentage | | Tree | | Acre | | (lbs/acre) | |
| 2-19 [symbol] | 6852 | [symbol] | 0.94 | [symbol] | 53.0 | d | 14.2 | a | 171 [symbol] | a | 171 [symbol] | a |
| Nonpareil-70 | 384 [symbol] | bc | 1.07 | cde | 64.6 | ab | 9.1 | bcd | 1101 | bcd | 1101 | bcd |
| Nonpareil-J | 3717 | bcd | 1.0 [symbol] | cde | 64.0 | abc | 8.6 | bcd | 106 [symbol] | bcd | 10 [symbol] [symbol] | bcd |
| Chips | 3623 | bcd | 1.02 | f | 53.8 | d | 8.1 | bcde | 985 | bcde | 985 | bcde |
| Kochi | 3134 | cd | 1.16 | b | 59.9 | c | 8.0 | cde | 965 | cde | 965 | cde |
| Nonpareil-7 | 32 [symbol] [symbol] | bcd | 1.08 | cde | [symbol] 5.1 | a | 7.8 | de | 941 | de | 941 | de |
| Kahl | 3139 | cd | 1.06 | ef | 47. [symbol] | [symbol] | 7.4 | d [symbol] | 8 [symbol] 9 | def | 8 [symbol] 9 | def |
| Year 30 | | | | | | | | | | | | |
| 2-19 [symbol] | 13149 | [symbol] | 0.78 | [symbol] | 54.3 | d | 22. [symbol] | [symbol] | 2756 | [symbol] | 4474 | a |
| Nonpareil-70 | 9340 | cde | 0.92 | bc | 66.3 | e | 18.9 | abcd | 2291 | abcd | 3393 | b |
| Kahl | 9594 | cd | 0.91 | bc | 47.6 | [symbol] | 19.3 | abcd | 2332 | abcd | 3222 | bcd |
| Nonpariel-J | 9137 | cde | 0.89 | bcd | 65.5 | a | 17.8 | bcde | 2152 | bcde | 3218 | bcd |
| Chips | 7681 | defg | 0. [symbol] 7 | cd | 54.4 | d | 14.7 | ef | 1780 | ef | 276 [symbol] | bcd |
| Kochi | 600 [symbol] | g | 1.0 [symbol] | [symbol] | 59.4 | bc | 14.3 | ef | 1729 | ef | 2694 | de |
| Year 31 | | | | | | | | | | | | |
| 2-19 [symbol] | 13472 | [symbol] | 0.93 | g | 54.3 | d | 27.4 | cd | 3321 | cd | 7795 | [symbol] |
| Nonpareil-70 | 12506 | bcd | 1.17 | cd | 66.3 | [symbol] | 30.7 | b | 3714 | b | 7106 | bc |
| Nonpariel-J | 11071 | d | 1.09 | cde | 65.5 | [symbol] | 26.6 | de | 3224 | de | 6442 | cd |
| Kahl | 10720 | de | 0.98 | fg | 47.6 | e | 22.6 | fg | 2733 | fg | 5954 | de |
| Chips | 11465 | cd | 0.97 | fg | 54.4 | d | 24.4 | ef | 2956 | ef | 5722 | ef |
| Kochi | 5882 | f | 1.28 | b | 69.5 | bc | 16.5 | h | 2002 | h | 469 [symbol] | g |
| Year 32 | | | | | | | | | | | | |
| 2-19 [symbol] | 14706 | [symbol] | 0.84 | f | 65.6 | f | 27.1 | c | 3285 | c | 11080 | a |
| Nonpareil-70 | 1375 [symbol] | ab | 1.04 | bcd | 74.6 | ab | 31.4 | [symbol] b | 3798 | [symbol] b | 10905 | abc |
| Nonpariel-J | 12 [symbol] 03 | abc | 1.04 | bcd | 71.6 | bcd | 29.0 | bc | 3513 | bc | [symbol] 955 | cd |
| Kahl | 11035 | cd [symbol] | 0. [symbol] 7 | ef | 59.1 | g | 21.1 | d [symbol] | 25 [symbol] 9 | d [symbol] | 8513 | ef |
| Chips | 9771 | ef | 0.93 | def | 58.6 | g | 20.0 | [symbol] | 2422 | [symbol] | [symbol] 144 | ef |
| Kochi | 7252 | g | 1.17 | [symbol] | [symbol] [symbol] .9 | de | 18.7 | [symbol] | 2259 | [symbol] | 6955 | h |
| Year 33 | | | | | | | | | | | | |
| Nonpareil-70 | 8823 | bcd | 1.28 | abcd | 72.3 | ab | 24.9 | [symbol] | 3011 | [symbol] | 13916 | ab |
| 2-19 [symbol] | 6833 | efg | 1.10 | bcdef | 56.1 | e | 16.7 | bc | 2020 | bc | 13100 | bc |
| Nonpariel-Jones | 8315 | cd [symbol] | 1.23 | abcdef | 70.9 | ab | 22.6 | [symbol] | 2737 | a | 12691 | c |
| Chips | 90 [symbol] 9 | abc | 1.15 | bcdef | 65.9 | abc | 23.0 | a | 27 [symbol] 9 | a | 10933 | d |
| Kahl | 7587 | cd [symbol] | 1.01 | f | 56.5 | d [symbol] | 16.9 | b | 2048 | c | 10561 | d |
| Kochi | 3902 | [symbol] | 1.40 | a | 64.4 | bcd | 12.1 | bc | 14 [symbol] 6 | bc | 8421 | [symbol] |
| Year 34 | | | | | | | | | | | | |
| Nonpareil-70 | 17744 | abc | 1.05 | bc | 70.7 | a | 41.0 | [symbol] | 49 [symbol] 2 | a | 1 [symbol] 878 | ab |
| 2-19 [symbol] | 18253 | ab | 0.91 | bcde | [symbol] 4.8 | abcd | 35.8 | a | 44 [symbol] 0 | a | 17560 | cd |
| Nonpariel-Jones | 1 [symbol] 993 | abcd | 0.96 | bcde | 70.0 | ab | 36.0 | [symbol] | 43 [symbol] 0 | [symbol] | 17051 | d |
| Chips | 11901 | f | 0.94 | bcde | 60.3 | de | 24.7 | bcd | 2985 | bcd | 13918 | e |
| Kahl | 12420 | f | 0.89 | cde | 53.5 | f | 24.4 | bcd | 2953 | bcd | 13514 | [symbol] |
| Kochi | [symbol] 701 | g | 1.22 | [symbol] | [symbol] 3.5 | cde | 23.3 | d | 2825 | d | 11247 | f |
| Year 35 | | | | | | | | | | | | |
| Nonpareil-70 | 8530 | b | 1.2 | bc | 70.9 | bc | 22.6 | ab | 2733 | ab | 21611 | [symbol] b |
| 2-19 [symbol] | 7617 | bc | 1.19 | bcd | 69.4 | bcd | 20.1 | abc | 2432 | abc | 20270 | bc |
| Nonpariel-Jones | 885 [symbol] | b | 1.18 | bcd | 67.7 | bcd | 23.0 | [symbol] b | 27 [symbol] 3 | ab | 19833 | c |
| Chips | 9008 | [symbol] | 0.92 | [symbol] | 75.3 | [symbol] b | 18.2 | bc | 2201 | bc | 1 [symbol] 41 [symbol] | d |
| Kahl | 8830 | b | 1.05 | fg | 55.0 | d | 20.4 | abc | 2465 | abc | 15979 | d |
| Kochi | 2025 | d | 1.41 | [symbol] | 26.0 | [symbol] | 6.3 | d | 783 | d | 12 [symbol] 18 | e |

[symbol]

indicates data missing or illegible when filed

What we claim is:

1. A new and distinct variety of almond tree designated ‘Kester’ as shown and described herein.
